(19) United States
(12) Patent Application Publication
(10) Pub. No.: US 2026/0166203 A1
(43) Pub. Date: Jun. 18, 2026

(54) COMPOSITE MATERIALS AND LAMINATES

(71) Applicant: W. L. Gore & Associates, Inc., Newark, DE (US)

(72) Inventors: Edward H. Cully, Clarksville, VA (US); Jeffrey B. Duncan, Flagstaff, AZ (US); Thomas R. McDaniel, Flagstaff, AZ (US); Rebecca L. Maryn, Flagstaff, AZ (US)

(21) Appl. No.: 19/139,588

(22) PCT Filed: Dec. 15, 2023
(86) PCT No.: PCT/US2023/084336

§ 371 Date: Jun. 16, 2025

Related U.S. Application Data
(60) Provisional application No. 63/433,143, filed on Dec. 16, 2022.

(51) Int. Cl. A61L 31/04; A61F 2/07; B32B 1/08; B32B 7/12; B32B 27/08; B32B 27/32

(52) U.S. Cl. CPC A61L 31/048; A61F 2/07; B32B 1/08; B32B 7/12; B32B 27/08; B32B 27/322; B32B 2250/05; B32B 2250/242; B32B 2305/026; B32B 2307/7376; B32B 2535/00

(57) ABSTRACT

An expandable sheath is disclosed herein, which has a first polymeric layer and a braided layer positioned radially outward of the first polymeric layer. The braided layer includes a plurality of filaments braided together. The expandable sheaths further include a resilient elastic layer positioned radially outward of the braided layer. The elastic layer is configured to apply radial force to the braided layer and the first polymeric layer. The expandable sheath further includes a second polymeric layer positioned radially outward of the elastic layer and bonded to the first polymeric layer such that the braided layer and the elastic layer are encapsulated between the first and second polymeric layers. Methods of making and using the devices disclosed herein are also disclosed, as are crimping devices that may be used in methods of making the devices disclosed herein.

### CROSS-REFERENCE TO RELATED APPLICATION

This application is a national phase application of PCT Application No. PCT/US 2023/084336, internationally filed on Dec. 15, 2023, which claims the benefit of Provisional Application No. 63/433, 143, filed Dec. 16, 2022, which are incorporated herein by reference in their entireties for all purposes.

### FIELD

The present disclosure relates generally to apparatuses, systems, and methods for providing composite materials operable to be wear-resistant. More specifically, the disclosure relates to apparatuses, systems, and methods that include wear-resistant materials that may be used in implantable medical devices.

### BACKGROUND

Materials used for manufacture are important as the materials provide specific qualities that are necessary for the article of manufacture to function for its intended purpose. Materials may be selected for various properties including, for example, longevity. Material selection is important in a variety of industries, including but not limited to the medical device industry and more specifically in implantable devices.

Implantable devices often need to remain within the body of a patient for a long period of time or be permanent. Often the implantable devices are positioned at locations that are subject to movement that may cause increased possibility of wear and/or failure due to certain movements, including repeated movements. The failure of implantable devices due to failure of the material can cause a number of adverse outcomes and therefore having materials that are capable to retaining structural integrity over a long period of time are important.

Many materials may be used across various industries and the same properties that are desirable in one industry may also be important in other industries.

### SUMMARY

Composite materials are provided herein that have high resistance to abrasion and wear during use. The composite materials achieve wear and abrasion resistance by combining the desirable qualities of different materials into a single composite material (e.g., a laminate) with a firm coupling of the different materials to each other to provide the wear-resistance qualities throughout.

According to one example (“Example 1”), a composite material is provided, the composite material including an expanded polytetrafluoroethylene layer; a densified expanded polytetrafluoroethylene layer coupled to the expanded polytetrafluoroethylene layer; a porous expanded polytetrafluoroethylene layer coupled to the densified expanded polytetrafluoroethylene layer; an adhesive; and a densified expanded polyethylene layer, the adhesive binding the expanded polyethylene layer to the porous expanded polytetrafluoroethylene layer.

According to another example (“Example 2”), further to Example 1, the adhesive is a low-density polyethylene.

According to another example (“Example 3”), further to Example 1, the composite material further includes a thin layer of low-density polyethylene coupled to the expanded polyethylene layer.

According to another example (“Example 4”), further to Example 3, the thin layer of low-density polyethylene and the adhesive sandwich the expanded polyethylene layer.

According to another example (“Example 5”), further to Example 1, the densified expanded polyethylene layer is from about 2 micrometers to about 5 micrometers thick.

According to another example (“Example 6”), further to Example 1, the adhesive is at least partially imbibed into the porous expanded polytetrafluoroethylene layer.

According to another example (“Example 7”), further to Example 6, the adhesive is processed at a range of about from 130 degrees Celsius to about 145 degrees Celsius to adhere the densified expanded polyethylene layer and the porous expanded polytetrafluoroethylene layer.

According to another example (“Example 8”), further to Example 1, the densified expanded polytetrafluoroethylene layer is from about 2 micrometers to about 10 micrometers thick.

According to another example (“Example 9”), further to Example 1, the porous expanded polytetrafluoroethylene layer is from about 10 micrometers to about 50 micrometers thick.

According to another example (“Example 10”), further to Example 1, the adhesive forms a layer that is from about 1 micrometers to about 200 micrometers thick.

According to an example (“Example 11”), an implantable device includes a tubular member formed of a composite material, the tubular member including: a first polymer layer including a blood contacting surface; a densified second polymer layer coupled to the first polymer layer; a porous, expanded third polymer layer coupled to the densified second polymer layer; an adhesive; and a densified expanded polyethylene layer, the adhesive binding the densified expanded polyethylene layer to the porous, expanded third polymer layer.

According to another example (“Example 12”), further to Example 11, the first polymer layer is an expanded polytetrafluoroethylene layer.

According to another example (“Example 13”), further to Example 11, the densified second polymer layer is a densified polytetrafluoroethylene layer.

According to another example (“Example 14”), further to Example 11, the porous, expanded third polymer layer is a porous, expanded polytetrafluoroethylene layer.

According to another example (“Example 15”), further to Example 11, the adhesive is a low-density polyethylene.

According to another example (“Example 16”), further to Example 15, the low-density polyethylene is imbibed into the porous, expanded third polymer layer.

According to another example (“Example 17”), further to Example 11, the tubular member is a graft.

According to another example (“Example 18”), further to Example 17, the implantable device further includes a stent coupled to the tubular member.

According to an example (“Example 19”), an implantable medical device, includes a first polytetrafluorethylene layer; and a second polyethylene layer coupled to the first polytetrafluoroethylene layer, wherein the second polyethylene layer is operable to be positioned against structures that are non-stationary relative to the second polyethylene layer.

According to another example (“Example 20”), further to Example 19, the first polytetrafluoroethylene layer and the second polyethylene layer are included in one of a graft, a tag, a cartilage replacement, an implantable medical device, and a removable medical device.

The foregoing Examples are just that, and should not be read to limit or otherwise narrow the scope of any of the inventive concepts otherwise provided by the instant disclosure. While multiple examples are disclosed, still other embodiments will become apparent to those skilled in the art from the following detailed description, which shows and describes illustrative examples. Accordingly, the drawings and detailed description are to be regarded as illustrative in nature rather than restrictive in nature.

### BRIEF DESCRIPTION OF THE DRAWINGS

The accompanying drawings are included to provide a further understanding of the disclosure and are incorporated in and constitute a part of this specification, illustrate embodiments, and together with the description serve to explain the principles of the disclosure.

FIG. 1 is an illustration of a cross section of a composite material including various layers of polytetrafluoroethylene and polyethylene, in accordance with an embodiment; and

FIG. 2 is an illustration of a cross section of another composite material including various layers of polytetrafluoroethylene including an outer layer of low-density polyethylene, in accordance with an embodiment;

FIGS. 3A-3E are images of wear abrasion of various composite materials, in accordance with various embodiments;

FIG. 4 is an illustration of an implantable medical device implementing a composite material, in accordance with an embodiment;

FIG. 5 is an illustration of an implantable medical device including composite material tags in positions of potential high abrasion, in accordance with an embodiment;

FIG. 6 is an implantable medical device formed of an expanded polytetrafluoroethylene and expanded polyethylene composite material, in accordance with an embodiment;

FIG. 7 is an implantable medical device including a graft attach tape formed of a composite material, in accordance with an embodiment;

### DETAILED DESCRIPTION

### Definitions and Terminology

This disclosure is not meant to be read in a restrictive manner. For example, the terminology used in the application should be read broadly in the context of the meaning those in the field would attribute such terminology.

With respect to terminology of inexactitude, the terms “about” and “approximately” may be used, interchangeably, to refer to a measurement that includes the stated measurement and that also includes any measurements that are reasonably close to the stated measurement. Measurements that are reasonably close to the stated measurement deviate from the stated measurement by a reasonably small amount as understood and readily ascertained by individuals having ordinary skill in the relevant arts. Such deviations may be attributable to measurement error, differences in measurement and/or manufacturing equipment calibration, human error in reading and/or setting measurements, minor adjustments made to optimize performance and/or structural parameters in view of differences in measurements associated with other components, particular implementation scenarios, imprecise adjustment and/or manipulation of objects by a person or machine, and/or the like, for example. In the event it is determined that individuals having ordinary skill in the relevant arts would not readily ascertain values for such reasonably small differences, the terms “about” and “approximately” can be understood to mean plus or minus 10% of the stated value.

As used herein, “couple” means join, connect, attach, adhere, affix, or bond, whether directly or indirectly, and whether permanently or temporarily.

The term “composite material” as used herein refers to a material including two or more material components with one or more different material properties from the other. In some examples, a composite material includes at least a first material component in the form of a membrane and a second material component in the form of a polymer that is combined with the membrane (e.g., by coating and/or imbibing processes). The term “laminate” as used herein refers to multiple layers of membrane, composite material, or other materials, such as, but not limited to a polymer, such as, but not limited to an elastomer, elastomeric or non-elastomeric material, and combinations thereof.

The term “film” as used herein generically refers to one or more of the membrane, composite material, or laminate.

The term “biocompatible material” as used herein generically refers to any material with biocompatible characteristics including synthetic materials, such as, but not limited to, a biocompatible polymer, or a biological material, such as, but not limited to, bovine pericardium. Biocompatible material may comprise a first film and a second film as described herein for various embodiments.

The term “polyethylene” (PE) as used herein is inclusive of all types of polyethylene, including but not limited to expanded polyethylene (ePE).

The term “polytetrafluoroethylene” (PTFE) as used herein is inclusive of all types of polytetrafluoroethylene, including but not limited to expanded polytetrafluoroethylene (ePTFE).

The term “graft attach tape” as used herein is inclusive of all types of tapes used to attached graft materials or as implemented onto a stent graft.

### Description of Various Embodiments

Persons skilled in the art will readily appreciate that various aspects of the present disclosure can be realized by any number of methods and apparatuses configured to perform the intended functions. It should also be noted that the accompanying drawing figures referred to herein are not necessarily drawn to scale, but may be exaggerated to illustrate various aspects of the present disclosure, and in that regard, the drawing figures should not be construed as limiting.

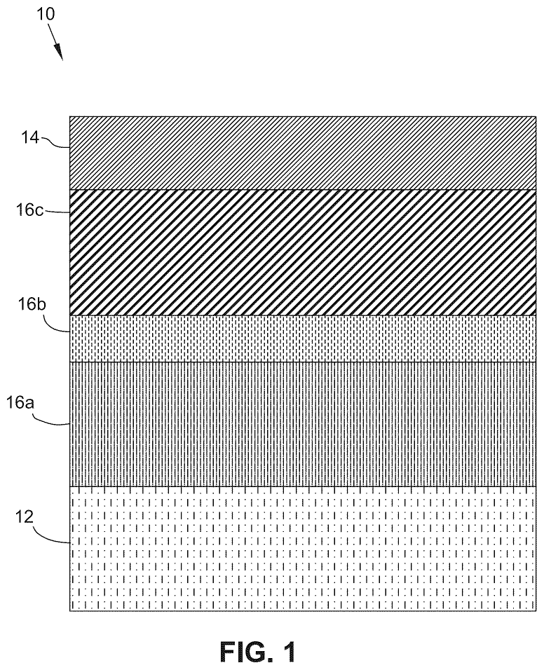

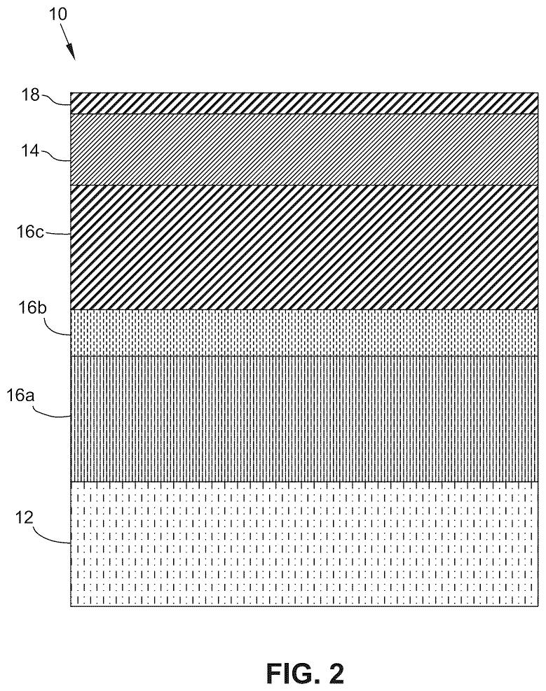

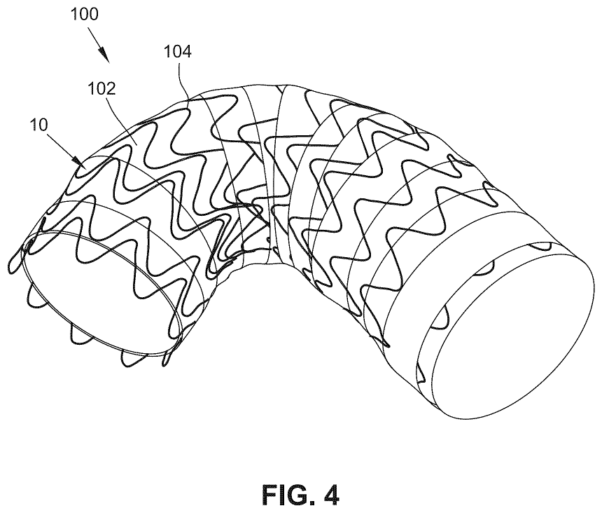

The composite material shown in FIG. 1 is provided as an example of the various features of the composite material and, although the combination of those illustrated features is clearly within the scope of invention, that example and its illustration is not meant to suggest the inventive concepts provided herein are limited from fewer features, additional features, or alternative features to one or more of those features shown in FIG. 1. For example, in various embodiments, the layers of the composite material shown in FIG. 1 may include the layers described with reference to FIG. 2. It should also be understood that the reverse is true as well. One or more of the components depicted in FIG. 1 can be employed in addition to, or as an alternative to components depicted in FIG. 2. The composite materials of FIGS. 1 and 2 may be implemented in a variety of devices and components such as those depicted in FIGS. 4-6. Although the devices depicted in FIGS. 4-6 may implement the composite materials described herein, it is understood that the composite material may be implemented with regard to various devices, systems, methods.

FIG. 1 illustrates an exemplary composite material 10 implementing various polymer layers to provide a durable, wear-resistant material that can be implemented in a variety of contexts, including but not limited to implantable medical devices. Various polymer layers are coupled together to provide specific properties to increase the durability against break-down of the material due to repeated movements that can result in wear of the material. Each layer may be implemented to provide various functions such as durability in a specific direction (e.g., X, Y, and Z or compression, tension, and so forth). By providing wear-resistant materials that are able to sustain repeated motions with limited degradation or damage, the longevity of implantable devices is increased which results in less procedures, decreased risk from failure of the devices, and increased confidence in the efficacy of the device.

The composite material 10 includes a plurality of layers. For example, the composite material 10 includes a base layer 12, an outer layer 14, and at least one intermediate layer 16. The base layer 12 and the outer layer 14 may include materials with properties suitable for implantation in a human, for example, materials that are biologically stable and suitable for contact or direct exposure to biological tissue. In some embodiments, the base layer 12 may include a material that is suitable for blood contact (e.g., polytetrafluoroethylene or polyethylene). The various layers may be coupled to each other in a variety of manners, including but not limited to bonding, adhesion, imbibing, and so forth. For example, the composite material 10 may include a first polymer layer (e.g., the base layer 12), a densified second polymer layer (e.g., a first intermediate layer 16a) coupled to the first polymer layer, a porous, expanded third polymer layer (e.g., a second intermediate layer 16b) coupled to the densified second layer, an adhesive (e.g., a third intermediate layer 16c), and a densified expanded polymer layer (e.g., the outer layer 14, wherein the outer layer is a formed of a different polymer than the base layer 12).

In a more specific example, the base layer 12 is a formed of polytetrafluoroethylene (PTFE) and the outer layer 14 is formed of polyethylene (PE).

The PTFE base layer 12 includes Z-axis strength and the PE outer layer 14 includes XY-axis strength which increases resistance to particulation of material when rubbed (which can result in release of loose material into the body as well as thinning of the material at the position where contact is occurring). The PTFE base layer 12 is operable to provide the Z-axis strength (e.g., via compression) to the PE outer layer 14 (which may have relatively less Z-axis strength) while the PE outer layer 14 limits damage from rubbing or abrasion).

The performance of the combined properties of the PTFE base layer 12 and the PE outer layer 14 are enhanced with a strong coupling of the PTFE base layer 12 and the PE outer layer 14. Intermediate layers 16 may be implemented to provide a strong bond to reduce the likelihood of delamination of the composite material 10 especially with materials that may include different material properties such as hydrophobicity. By including a strong bond between the layers, each of the layers imparts their individual effect to the combined substrate as a whole. The intermediate layer may also impart other characteristics to the substrate, including but not limited to z-axis strength. Any number of intermediate layers may be implemented in order to achieve strong bonding as well as providing other properties including increased strength and durability.

Referring further to the example including the PTFE base layer 12 and the PE outer layer 14, the intermediate layers 16 may include layers formed of the same or similar materials as one or both of the PTFE base layer 12 and the PE outer layer 14. In other embodiments, the intermediate layers 16 may include layers formed of different materials as the PTFE base layer 12 and the PE outer layer 14, the different materials being able to bond strongly with one or both of the PTFE base layer 12 and the PE outer layer 14.

In one example, FIG. 1 represents a composite material 10 with the PTFE base layer 12, the PE outer layer 14, and both PTFE and PE intermediate layers 16. For example, the PTFE base layer 12 is formed of expanded PTFE (ePTFE) and the PE outer layer 14 is formed of expanded polyethylene (ePE), with the intermediate layers 16 including a densified ePTFE intermediate layer 16a, an ePTFE intermediate layer 16b, and a low-density PE (LDPE) intermediate layer 16c (implemented as an adhesive). In this example, the ePTFE base layer 12 may be a blood contacting surface as the properties of ePTFE are stable for blood contact, the densified ePTFE intermediate layer 16a bonded to the ePTFE base layer 12 may provide Z-axis strength to the composite material 10, the ePTFE intermediate layer 16b bonded to the densified ePTFE intermediate layer 16a, the ePTFE intermediate layer 16b including a porous, open network, the LDPE intermediate layer 16c being bonded to the ePTFE intermediate layer 16b, and the PE outer layer 14 being bonded to the LDPE intermediate layer 16c. The LDPE intermediate layer 16c acts as an adhesive between the PE outer layer 14 and the ePTFE intermediate layer 16b. For example, the LDPE intermediate layer 16c may be imbibed into the ePTFE intermediate layer 16b. Because of the open, porous properties of the ePTFE intermediate layer 16b, the LDPE intermediate layer 16c is able to at least partially penetrate or be imbibed into the pores of the ePTFE intermediate layer 16b, which can occur at a low processing temperature (e.g., about 130 degrees Celsius). In some embodiments, the PE outer layer 14 can be a densified ePE material that is resistant to abrasion. The PE outer layer 14 may be applied to the LDPE intermediate later 16c at a low processing temperature (e.g., about 130 degrees Celsius). It is understood that various types of films may be implemented for the base, intermediate, and outer layers. For example, the layers may be reversed such that PE is implemented on the base layer 12 and ePTFE is implemented on the outer layer 14. It is understood that the layers discussed herein may include one or more layers (for example, a layer may include a plurality of films of the same or similar materials coupled together to form an individual layer).

Referring still to FIG. 1, the densified ePTFE intermediate layer 16a may include a densified ePTFE film that is operable to provide circumferential and creep strength to the composite material 10. For an example of the densified ePTFE film, see U.S. Pat. No. 7,521,010 granted to Kennedy and Hollenbaugh Jr. The densified ePTFE intermediate layer 16a may be about 1 micrometers to about 200 micrometers thick. In some embodiments, the intermediate layer 16a is from about 1 micrometers to about 2 micrometers, from about 2 micrometers to about 3 micrometers, from about 3 micrometers to about 4 micrometers, from about 4 micrometers to about 5 micrometers, from about 5 micrometers to about 6 micrometers, from about 6 micrometers to about 7 micrometers, from about 7 micrometers to about 8 micrometers, from about 8 micrometers to about 9 micrometers, from about 9 micrometers to about 10 micrometers, from about 10 micrometers to about 15 micrometers, from about 15 micrometers to about 20 micrometers, from about 20 micrometers to about 25 micrometers, from about 25 micrometers to about 30 micrometers, from about 30 micrometers to about 35 micrometers, from about 35 micrometers to about 40 micrometers, from about 40 micrometers to about 45 micrometers, from about 45 micrometers to about 50 micrometers, from about 50 micrometers to about 100 micrometers, from about 100 micrometers to about 150 micrometers, and from about 100 micrometers to about 200 micrometers. In one example the densified ePTFE intermediate layer 16a is formed of densified ePTFE with fluorinated ethylene propylene (FEP) used as an adhesive which is operable to maintain a solid bond or adhesion.

The ePTFE intermediate layer 16b may be formed of an open, porous ePTFE material. The ePTFE intermediate layer 16b provide an attachment surface into which other layers (e.g., the LDPE intermediate layer 16c) may be imbibed. The ePTFE intermediate layer 16b may also provide burst strength, suture retention strength, longitudinal strength, and creep resistance to the overall construct (e.g., the composite material 10). The ePTFE intermediate layer 16b may be about 1 micrometers to about 200 micrometers thick. In some embodiments, the ePTFE intermediate layer 16b is from about 1 micrometers to about 2 micrometers, from about 2 micrometers to about 3 micrometers, from about 3 micrometers to about 4 micrometers, from about 4 micrometers to about 5 micrometers, from about 5 micrometers to about 6 micrometers, from about 6 micrometers to about 7 micrometers, from about 7 micrometers to about 8 micrometers, from about 8 micrometers to about 9 micrometers, from about 9 micrometers to about 10 micrometers, from about 10 micrometers to about 15 micrometers, from about 15 micrometers to about 20 micrometers, from about 20 micrometers to about 25 micrometers, from about 25 micrometers to about 30 micrometers, from about 30 micrometers to about 35 micrometers, from about 35 micrometers to about 40 micrometers, from about 40 micrometers to about 45 micrometers, from about 45 micrometers to about 50 micrometers, from about 50 micrometers to about 100 micrometers, from about 100 micrometers to about 150 micrometers, and from about 100 micrometers to about 200 micrometers. Some embodiments of the ePTFE intermediate layer 16b may include FEP as an adhesive within individual films comprising the ePTFE intermediate layer 16b.

The LDPE intermediate layer 16c acts as an adhesive to bind various layers together. In some embodiments, the LDPE intermediate layer 16c forms a layer that is from 01 micrometers to about 200 micrometers thick. In some embodiments, the LDPE intermediate layer 16c is from about 1 micrometers to about 2 micrometers, from about 2 micrometers to about 3 micrometers, from about 3 micrometers to about 4 micrometers, from about 4 micrometers to about 5 micrometers, from about 5 micrometers to about 6 micrometers, from about 6 micrometers to about 7 micrometers, from about 7 micrometers to about 8 micrometers, from about 8 micrometers to about 9 micrometers, from about 9 micrometers to about 10 micrometers, from about 10 micrometers to about 15 micrometers, from about 15 micrometers to about 20 micrometers, from about 20 micrometers to about 25 micrometers, from about 25 micrometers to about 30 micrometers, from about 30 micrometers to about 35 micrometers, from about 35 micrometers to about 40 micrometers, from about 40 micrometers to about 45 micrometers, from about 45 micrometers to about 50 micrometers, from about 50 micrometers to about 100 micrometers, from about 100 micrometers to about 150 micrometers, and from about 100 micrometers to about 200 micrometers. The LDPE intermediate layer 16c may also be operable to provide some stiffness as a backer to the other layers that results in increased wear resistance and puncture/tear resistance of the composite material 10. Although the example provided with respect to FIG. 1 is formed of LDPE, it is understood that the intermediate layer 16c may also be formed of various other materials acting as an adhesive. For example, the intermediate later 16c may include a fluoropolymer, a polyurethane copolymer, a silicone wet lay-up, polyurethane, and so forth. Such adhesives may be selected for the low temperature at which they melt, thus allowing the adhesive to be applied without disrupting the properties of other layers via the thermal process.

In some embodiments, the materials discussed herein may be selected based on their molecular weight, which may be a strong indicator for strength and density and is understood to play a role in toughness or compressibility. It is also understood that the materials being implemented may be selected for the pre-processing that has occurred including heat treatments prior to incorporation in the composite as well as how it is affected (e.g., via heat treatment) during the build of the composite. It is also understood that the temperature at which the materials are implemented may have an effect on the properties of the composite material (e.g., level of creep resistance at room temperature vs. body temperature).

FIG. 2 is an example of a composite material 10 similar to the composite material discussed with respect to FIG. 1, but further includes an LDPE layer 18 coupled to the PE outer layer 14, such that the PE outer layer 14 is sandwiched between two LDPE layers. It is understood that various layers may be positioned as the base layer 12 and the outer layer 14 according to specific properties that may be desirable to include on the surface. Various features that may be selected for include porosity, texture, biocompatibility, coatability, treatability, coefficient of friction, and so forth. In some embodiments, the PE outer layer 14 (e.g., densified ePE) is from 2 micrometers to about 10 micrometers thick.

The various layers may include similar thicknesses, may each have different thicknesses, or a combination of similar and different thicknesses. For example, in some embodiments, the ePTFE layers may be an ultra-thin wall ePTFE layer. In some embodiments, the PTFE base layer 12 (e.g., densified ePTFE) is from 10 micrometers to about 50 micrometers thick. The PTFE base layer 12 may be provided in a variety of configurations, including but not limited to a tubular shape or a sheet. When formed into a tubular shape, the PTFE base layer 12 may provide a seamless tube. The PTFE base layer 12 may provide longitudinal strength to the composite material 10. The PTFE base layer 12 provides a porous luminal surface for tissue ingrowth and thrombus resistance (e.g., via patency).

In some embodiments, the PE outer layer 14 may be either a gel or paste processed ePE film. The PE outer layer 14, as discussed, may be coated with LDPE on one or both sides to provide increased adhesion to other layer and strength to the composite material 10. For example, when the PE outer layer 14 is coated with LDPE on the outer surface, the LDPE is operable to provide wear durability by helping reduce surface tearing and/or disruptions (e.g., holds the surface intact).

In some embodiments, the PE layer can be bonded to the PTFE layer by each layer respectively having at least one of the surfaces imbibed with LDPE. Once the PE layer and the PTFE layer have a surface that is imbibed with LDPE, the imbibed surfaces of the PE and the PTFE are bonded together (e.g., via thermal treatment at about 130 degrees Celsius). The bonding between the LDPE imbibed surfaces of the PE and PTFE layers can result in about a 4 Newton peel strength, which indicates a strong bond between the two layers. A stronger bond between the various layers results in the individual properties of the various layers to be incorporated into the composite material 10. For example, when a strong bond is achieved between PTFE and PE, the Z-axis strength of the PTFE layer is incorporated in the composite material and the resistance to pilling (e.g., material being pushed out of place resulting in a thinner layer) of the PE layer is also incorporated in the composite material. When the bond between the PE layer and the PTFE layer is compromised or is not complete (e.g., in composite materials with lower peel strength), the Z-axis strength of the composite material 10 decreases and may result in material stretching and/or ripping and tearing of the composite material 10.

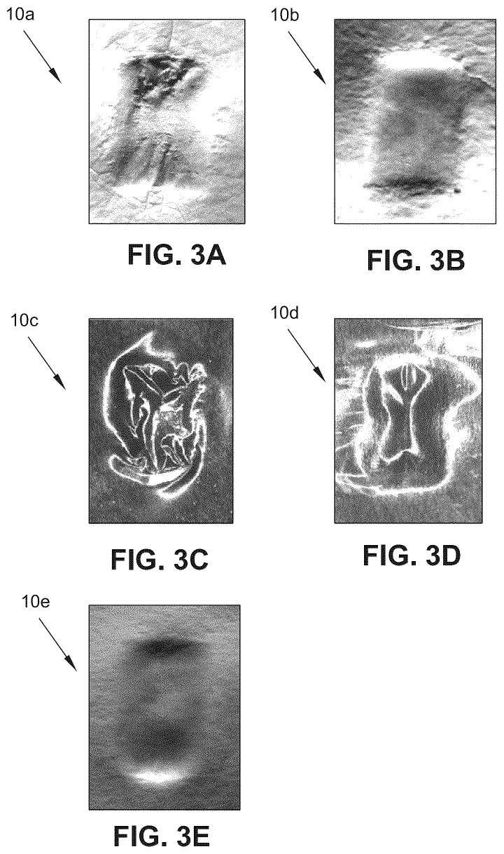

FIGS. 3A-3E depict wear resistance testing results of various composite materials. The wear resistance test implemented included providing a 2 Newton force a 5 Hz for a 300 sec interval with a Stop Test at 1500 cycles. FIG. 3A depicts an ePE membrane (composite material 10A) with a tight porous structure (e.g., pore size greater than 5 micrometers. The ePE membrane is not treated with LDPE. As depicted, the ePE membrane experiences pilling and tearing of a top layer and shows failure by 1500 cycles. FIG. 3B depicts an ePE membrane with a layer of LDPE (composite material 10b), the ePE membrane having a tight porous structure (e.g., about 3 micrometers). By including LDPE with the ePE membrane, pilling was reduced, no tearing occurred, and a relatively smoother surface was retained after the testing cycles. FIG. 3C depicts a densified ePE membrane with a layer of LDPE (composite material 10c), the densified ePE membrane (ADD measurement). This resulted in no piling, ripping or tearing. The densified ePE membrane showed signs of some material stretching in the Z-axis. FIG. 3D depicts a composite material 10d with two densified ePE membranes with a layer of LDPE. The composite material 10d, when subjected to the wear testing, resulted in no piling, ripping or tearing. The composite material 10d showed signs of some material stretching in the Z-axis with less stretching relative to the composite material of FIG. 3C. FIG. 3E depicts a composite material 10e with a densified ePE membrane and an ePE layer (having a tight porous structure) with a layer of LDPE. The composite material 10e, when subjected to the wear testing, resulted in no piling, ripping or tearing. The composite material 10e showed minimal material stretching in the Z-axis and fully preserving the ePE layer.

The various layers of the composite material described herein may be processed individually or in combination. Various processes may be implemented, including but not limited to those described in U.S. Pat. No. 9,926,416 to Sbriglia which issued Mar. 27, 2018, U.S. Pat. No. 10,577,468 to Sbriglia which issued Mar. 3, 2020, PCT Publication No. WO 2020/028328 by Bell which was filed Jul. 30, 2019, and PCT Publication No. WO 2020/028331 by Bell which was filed Jul. 30, 2019.

### EXAMPLES

The composite material 10 discussed herein may be implemented on a variety of devices and in a variety of contexts. Examples are provided below in which the composite material 10 may be implemented. The examples below are not to be construed as limiting.

### Example 1

The composite material 10 may be implemented with an implantable stent graft 100. Referring to FIG. 4, the stent graft 100 includes a graft member 102 and a stent member 104 coupled thereto. The graft member 102 is formed of the composite material 10. The composite material 10 is formed as a tubular member, the graft member 102 including the PTFE base layer 12 as a blood contacting surface and the PE outer layer 14 coupled to the stent member 104 (see FIGS. 1 and 2). The PE outer layer 14 is operable to resist wear due to movement of the stent member 104 during implantation and use. This is relevant in many contexts including when the stent graft 100 is implanted in a curved vessel (e.g., in or proximate the aortic arch) or in a vessel with high pressures or repeated movement (e.g., the aorta). By implementing the composite material 10 discussed, repeated movements are less likely to result in wear through the graft member 102 of the stent graft 100.

A biocompatible material for the graft components, discussed herein, may be used in addition to the composite material 10 as discussed herein or used in combination or as a layer of the composite material 10. In certain instances, the graft may include a fluoropolymer. In some instances, the graft may be formed of, such as, but not limited to, a polyester, a silicone, a urethane, a polyethylene terephthalate, or another biocompatible polymer, or combinations thereof. In some instances, bioresorbable or bioabsorbable materials may be used, for example a bioresorbable or bioabsorbable polymer. In some instances, the graft can include Dacron, polyolefins, carboxy methylcellulose fabrics, polyurethanes, or other woven, non-woven, or film elastomers.

In addition, nitinol (NiTi) may be used as the material of the frame or stent (and any of the frames discussed herein), but other materials such as, but not limited to, stainless steel, L605 steel, polymers, MP35N steel, polymeric materials, Pyhnox, Elgiloy, or any other appropriate biocompatible material, and combinations thereof, can be used as the material of the frame. The super-elastic properties and softness of NiTi may enhance the conformability of the stent. In addition, NiTi can be shape-set into a desired shape. That is, NiTi can be shape-set so that the frame tends to self-expand into a desired shape when the frame is unconstrained, such as when the frame is deployed out from a delivery system.

### Example 2

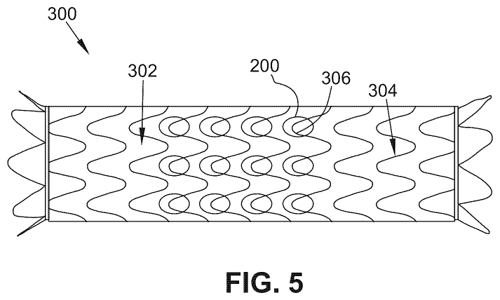

The composite material may be implemented as a patch or tag 200 on implantable medical devices at positions where repeated movements can result in wear to a surface of the implantable medical device. As illustrated in FIG. 5, a stent graft 300 is provided with tags 200 positioned at positions of potential wear due to abrasion. For example, the stent graft includes a stent member 302 and a graft member 304 coupled together. The stent member 302 includes a plurality of apices 306. The tags 200 are positioned between the apices 306 of the stent member 302 and the graft member 304 (e.g., the abluminal surface of the graft member 304). The tags 200 may be coupled to the graft member 304 via a variety of methods. For example, the tags 200 may be attached via an adhesive or applied via a thermal process (e.g., a heat gun). In other embodiments the tag can be formed of PE or IPA that is applied as a slurry throughout mask (e.g., imbibed). It is understood that the tags 200 may be applied at various positions on the stent graft 300 at which wear or abrasion may occur.

### Example 3

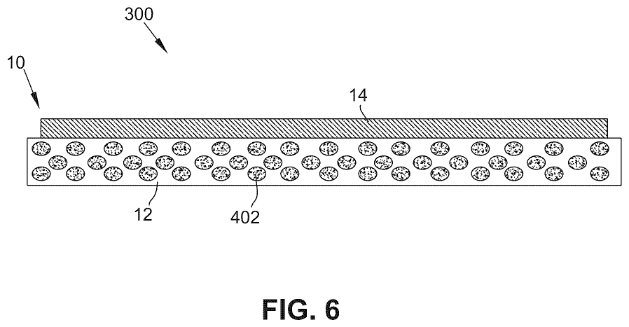

Referring to FIG. 6, a prosthetic cartilage 400 is illustrated. The prosthetic cartilage 400 is formed of the composite material 10. The composite material includes the PE outer layer 14 and the PTFE base layer 12. The PTFE base layer 12 may include a hydrogel 402 that help provide a cushioning function to the composite material 10. The PE outer layer 14 provides the high abrasion or wear resistance as discussed. The prosthetic cartilage 400 may be implemented in a variety of positions, including but not limited to being used as a replacement prosthetic meniscus

### Example 4

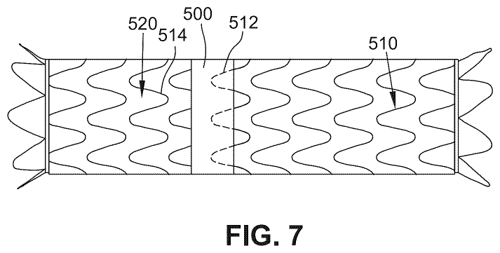

Referring to FIG. 7, a graft attach tape 500 is provided using the composite materials 10 discussed herein. For example, the graft attach tape 500 may be implemented on a stent graft, where the stent structure 510 includes first apices 512 and second apices 514. The graft attach tape 500 may be implemented to attach the first apices 512 to graft material 520. In some embodiments, the graft attach tape 500 may include a width such that the graft attach tape is positioned between the second apices 514 and the graft material 520, thus providing a wear resistance barrier between the second apices 514 and the graft material 520. For example, the graft attach tape 500 may be implemented for ultra-thin wall devices that may benefit from increased wear resistance at positions that may experience increased wear during use. FIG. 7 is shown with only one row of the apices 512, 514 with the graft attach tape 500 for clarity, it is understood that the graft attach tape 500 could be positioned to cover or be positioned between the apices 512, 514 of the device.

The invention of this application has been described above both generically and with regard to specific embodiments. It will be apparent to those skilled in the art that various modifications and variations can be made in the embodiments without departing from the scope of the disclosure. Thus, it is intended that the embodiments cover the modifications and variations of this invention provided they come within the scope of the appended claims and their equivalents.

1. A composite material, comprising:
an expanded polytetrafluoroethylene layer;
a densified expanded polytetrafluoroethylene layer coupled to the expanded polytetrafluoroethylene layer;
a porous expanded polytetrafluoroethylene layer coupled to the densified expanded polytetrafluoroethylene layer;
an adhesive; and
a densified expanded polyethylene layer, the adhesive binding the expanded polyethylene layer to the porous expanded polytetrafluoroethylene layer.

2. The composite material of claim 1, wherein the adhesive is a low-density polyethylene.

3. The composite material of claim 1, further comprising a thin layer of low-density polyethylene coupled to the expanded polyethylene layer.

4. The composite material of claim 3, wherein the thin layer of low-density polyethylene and the adhesive sandwich the expanded polyethylene layer.

5. The composite material of claim 1, wherein the densified expanded polyethylene layer is from about 2 micrometers to about 5 micrometers thick.

6. The composite material of claim 1, wherein the adhesive is at least partially imbibed into the porous expanded polytetrafluoroethylene layer.

7. The composite material of claim 6, wherein the adhesive is processed at a range of about from 130 degrees Celsius to about 145 degrees Celsius to adhere the densified expanded polyethylene layer and the porous expanded polytetrafluoroethylene layer.

8. The composite material of claim 1, wherein the densified expanded polytetrafluoroethylene layer is from about 2 micrometers to about 10 micrometers thick.

9. The composite material of claim 1, wherein the porous expanded polytetrafluoroethylene layer is from about 10 micrometers to about 50 micrometers thick.

10. The composite material of claim 1, wherein the adhesive forms a layer that is from about 1 micrometers to about 200 micrometers thick.

11. An implantable device comprising:
a tubular member formed of a composite material, the tubular member including:
a first polymer layer including a blood contacting surface;
a densified second polymer layer coupled to the first polymer layer;
a porous, expanded third polymer layer coupled to the densified second polymer layer;
an adhesive; and
a densified expanded polyethylene layer, the adhesive binding the densified expanded polyethylene layer to the porous, expanded third polymer layer.

12. The implantable device of claim 11, wherein the first polymer layer is an expanded polytetrafluoroethylene layer.

13. The implantable device of claim 11, wherein the densified second polymer layer is a densified polytetrafluoroethylene layer.

14. The implantable device of claim 11, wherein the porous, expanded third polymer layer is a porous, expanded polytetrafluoroethylene layer.

15. The implantable device of claim 11, wherein the adhesive is a low-density polyethylene.

16. The implantable device of claim 15, wherein the low-density polyethylene is imbibed into the porous, expanded third polymer layer.

17. The implantable device of claim 11, wherein the tubular member is a graft.

18. The implantable device of claim 17, further comprising a stent coupled to the tubular member.

19. An implantable medical device, comprising:
a first polytetrafluorethylene layer; and
a second polyethylene layer coupled to the first polytetrafluoroethylene layer, wherein the second polyethylene layer is operable to be positioned against structures that are non-stationary relative to the second polyethylene layer.

20. The implantable medical device of claim 19, wherein the first polytetrafluoroethylene layer and the second polyethylene layer are included in one of a graft, a tag, a cartilage replacement, an implantable medical device, and a removable medical device.
